(19) United States
(12) Patent Application Publication
(10) Pub. No.: US 2024/0173256 A1
(43) Pub. Date: May 30, 2024

(54) HDL MIMICKING TARGETED DRUG DELIVERY SYSTEM FOR THE TREATMENT OF SOLID TUMORS

(71) Applicant: University of North Texas Health Science Center, Fort Worth, TX (US)

(72) Inventors: Nirupama SABNIS, Fort Worth, TX (US); Andras LACKO, Fort Worth, TX (US); Rafal FUDALA, Fort Worth, TX (US)

(73) Assignee: University of North Texas Health Science Center, Fort Worth, TX (US)

(21) Appl. No.: 18/522,220

(22) Filed: Nov. 28, 2023

Related U.S. Application Data
(60) Provisional application No. 63/385,293, filed on Nov. 29, 2022.

(51) Int. Cl. A61K 9/107; A61K 31/704; A61K 47/12; A61K 47/24; A61K 47/28; A61K 47/42; A61K 51/12

(52) U.S. Cl. CPC A61K 9/1075; A61K 31/704; A61K 47/12; A61K 47/24; A61K 47/28; A61K 47/42; A61K 51/1227

(57) ABSTRACT

The present disclosure is directed to an SR-B1-targeting nanomicelle and compositions, methods of synthesis and methods of use thereof. The nanomicelle may be an HDL-mimetic drug delivery system and may be cross-linked with DSS. The composition may contain a therapeutic agent with the ability to treat cancers, especially sarcomas.

This application claims the benefit of U.S. Provisional Patent Application No. 63/385,293, filed Nov. 29, 2022, the entirety of which is incorporated herein by reference.

This application contains a Sequence Listing XML, which has been submitted electronically and is hereby incorporated by reference in its entirety. Said XML Sequence Listing, created on Nov. 28, 2023, is named UNTXP0012US.xml and is 1,971 bytes in size.

### BACKGROUND

### I. Field of the Disclosure

The present disclosure relates to the fields of medicine, pharmacology and molecular biology. In particular, the disclosure relates to drug delivery systems for treating cancers, such as sarcomas.

### II. Related Art

Sarcomas account for about 13% of all cancers in patients under 20 years of age and are associated with a comparatively poor prognosis. While, the 5-year survival rate for most pediatric cancers is nearly 80%, the survival for sarcomas is about 60% to 70% (Williams et al., 2016). The prognosis for children with metastatic sarcomas drops to 20-30% overall survival rates. Current efforts to develop improved treatments, have so far fallen short of providing improved outcomes for pediatric patients with non-metastatic disease, despite the aggressive, multi-agent strategies applied (Anderson et al., 2015). Consequently, new approaches are urgently needed to improve the prognosis, especially for children and young adults with sarcomas.

While anthracycline drugs, especially doxorubicin (DOX), have been utilized frequently and successfully in anti-cancer chemotherapy, drug resistance and dose dependent cardiotoxicity have posed major challenges to their therapeutic efficacy (McGowan et al., 2017). In an effort to improve the clinical efficacy of anthracyclines, several DOX derivatives have been developed. Among these, N-Benzyladriamycin-14-valerate (AD 198, see FIG. 1), has shown the potential to reduce (off target) cardiotoxicity (Edwards et al., 2013).

While using liposomes (Solomon et al., 2008) and prodrugs (Markovic et al., 2020) produced improved solubility, bioavailability and survival in animal models, earlier studies suggest that substantial additional therapeutic benefits are likely to accrue via the application of high-density lipoprotein (HDL) type nanoparticles (NPs) as drug transporters in Ewing sarcoma (EWS) therapeutics (Yang et al., 2021; Bell et al., 2018). Several human studies support the role of the scavenger receptor class B type I (SR-B1) receptor in carcinogenesis and survival of cancer cells, as evidenced by the frequently reported reduced plasma cholesterol (particularly HDL cholesterol) in cancer patients. Studies suggest that lipoprotein receptors (especially the SR-B1/HDL receptor) are highly active on the surface of malignant cells and thus can be used to facilitate the delivery of anti-cancer agents (Mooberry et al., 2016). Additional research studies showed that ablation of the SR-B1 receptor in breast cancer cells led to a decrease in cell proliferation, migration and invasion, leading to reduced viability of prostate cancer cells (Karimi et al., 2021).

Regarding nano-delivery systems, Wilhelm et al. (2016) showed that on the average, only 0.7% of the injected therapeutic nanoparticles actually find their way into the tumor. These findings identified the challenges facing nano-delivery agents to reach the intended target sites. Both the rHDL (reconstituted HDL) NPs and the next generation, MYR-5A NPs are built on the inherent properties of circulating HDL particles to structurally accommodate a broad range of anti-cancer agents (Lacko et al., 2015) and subsequently, selectively, deliver them to cancer cells and tumors via the same non-endocytic mechanism that is known for delivering cholesteryl esters to target cells (Meyer et al., 2014). This mechanism is likely to prevent drug resistance and is known to facilitate endosomal escape (Maugeri et al., 2019). In addition, the rHDL NPs and their constituents have already been tested in several cardio-vascular clinical trials without immune reactions or toxic side effects. Enhanced therapeutic benefits are also anticipated while utilizing HDL type NPs, via the efficient delivery of anti-cancer agents, due to their small size (˜20 nanometer diameter), extended circulation time, and superior biocompatibility (Kuai et al., 2016).

In previous studies, the inventors evaluated the spectroscopic properties of the 5A (DWLKAFYDKVAEKLKEAF-P-DWAKAAYDKAAEKAKEAA; SEQ ID NO: 1) and MYR-5A peptides to gain structural insights into the assembly of MYR-5A NPs. HDL-based drug formulations have been well documented to increase the bioavailability and the therapeutic efficacy of anti-cancer (and other) drugs, via increasing their solubility in water-based media and promotion of their delivery to cancer cells and tumors via a selectively targeted receptor-mediated uptake mechanism (McMahon et al., 2015). Recently, a new class of HDL mimetics has emerged, involving peptides with amino acid sequences that resemble the primary structure of the amphipathic alpha helices within the Apo A-I protein (Fukuhara et al., 2014). Based on the observations of the inventors and published work by others, EWS cell lines and EWS patient tumor samples show robust overexpression of the SR-B1 receptor, while cardiomyocytes and most other non-malignant cells show limited or no detectable expression. Consequently, the SR-B1 targeted approach has the capability to identify patients with the most aggressive type of tumors, as candidates for a personal approach and thus lead to the appropriate therapy and improved patient outcomes.

### SUMMARY

Thus, in accordance with the present disclosure, there is provided a composition comprising a self-assembling nanomicelle comprised of a scavenger receptor class B type 1 (SR-B1) ligand cross-linked with disuccinimidyl suberate (DSS). The ligand may be a recombinant high-density lipoprotein, or a myristic acid conjugated-5A peptide. The composition may further comprise sphingomyelin and/or cholesterol-oleate and/or a therapeutic agent, such as a small molecule, a peptide or protein, a nucleic acid, a toxin, or a radioactive compound.

The composition may be formulated as a unit dose and or formulated for systemic administration. The composition may be formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

Also provided is A method of delivering a payload to a cell expressing a scavenger receptor class B type 1 (SR-B1) comprising contacting said cell with a composition as defined herein. The payload may be a small molecule, such as an anthracycline, such as a doxorubicin or doxorubicin derivative. The doxorubicin derivative may be N-benzyladriamycin-14-valerate. The composition may be contacted with said cell in vitro or in vivo. The method, prior to said contacting, may be characterized as involving a cell that exhibits aberrant expression or activity of SR-B1.

In another embodiment, there is provided a pharmaceutical formulation comprising the composition as defined herein, wherein the formulation further comprises an excipient. The excipient may comprise a buffer, such as a phosphate buffer. The excipient may comprise a salt, such as sodium chloride. The excipient may comprise phosphate buffered saline.

Further provided is a kit comprising the composition as defined herein. The kit may be for use in the treatment of a cancer, such as a solid tumor, such as a sarcoma, such as Ewing sarcoma.

In yet another embodiment, there is provided a method of treating a disease or disorder in a patient comprising administering to the patient in need thereof a therapeutically effective amount of the composition as defined herein. The disease or disorder may be cancer, such as a solid cancer, such as a carcinoma, sarcoma, lymphoma, melanoma, mesothelioma, multiple myeloma, or seminoma. The sarcoma may be Osteosarcoma, Chondrosarcoma, Ewing Sarcoma, Rhabdomyosarcoma, Leiomyosarcoma, Angiosarcoma, Fibrosarcoma/Myofibrosarcoma, Chordoma or Liposarcoma. The sarcoma may be a pediatric sarcoma, such as is Osteosarcoma,

The method may comprise administering the compound once or administering the compound two or more times. The method may further comprise a second cancer therapy, such as surgery, a second chemotherapeutic agent, a radiotherapy, or an immunotherapy. The second chemotherapeutic agent may be an alkylating agent or an alkylating-like agent, may be ifosfamide or olaratumab, may be a radiotherapy, such as x-ray therapy.

In yet a further embodiment, there is provided a method of synthesizing the composition of claim 1, the method comprising (a) contacting the SR-B1 ligand with a therapeutic agent to form a reaction mixture; (b) dialyzing the reaction mixture against a buffer; (c) contacting the SR-B1 ligand with a DSS crosslinker to form a second reaction mixture; and (d) incubating the second reaction mixture for a sufficient time to produce the composition. The reaction mixture may further comprise a buffer, such as phosphate buffered saline. The reaction mixture may further comprise an organic solvent, such as ethanol, such as where the ratio of a buffer to an organic solvent is from about 5:1 to about 1:1 or is from about 4:1 to about 2:1 or is about 3:1.

The method may comprise the use of a microfluidic technology, such as one that employs a Nanoassemblr instrument. The dialysis buffer may comprise phosphate buffered saline. The second reaction mixture may be incubated for at least 2 minutes, or for at least 10 minutes, or for about 30 minutes.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number. It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Conceptual Model of Myr 5A-AD 198 Nanoparticle.

FIG. 2: Determination of Critical micellar concentration using Fluorescence Intensity Ratio (373 nm/384 nm) of Different Concentrations of MYR-5A.

FIG. 3: FPLC Chromatogram for MYR-5A-AD 198 and Plasma.

FIG. 4: FPLC Chromatogram for MYR-5A-AD 198 Nanoparticles Incubated in Plasma at Different Time Intervals.

FIG. 5: MYR-5A-AD 198 Retention of AD 198 in Human Plasma Over Time.

FIG. 6: Drug Retention of MYR-5A-AD 198 Over Time with Different Cross Linkers.

FIG. 7: Encapsulation Efficiency of MYR-5A-AD 198 with different ratio of Myr 5A to DSS.

FIG. 8A-8B: Encapsulation Efficiency of MYR-5A-AD 198 with Different Ratios of MYR-5A and AD 198. FIG. 8A—Encapsulation Efficiency. FIG. 8B—Anisotropy of MYR-5A-AD 198.

FIG. 9: FPLC fraction analysis of cross linked Myr 5A-AD 198 Nanoparticles for protein and drug.

FIG. 10: Transmission Electron micrograph of cross linked Myr 5A-AD 198 Nanoparticles.

FIG. 11: Expression of the SR-B1 receptor by EWS cell lines and cardiomyocytes determined via flow cytometry.

FIGS. 12A-12C: FIG. 12A—Uptake of Radioactive Cholesterol Oleate 3[H] in EWS Cell Line A673 in Presence of SR-B 1 Antibody. FIG. 12B—Uptake of Radioactive Cholesterol Oleate 3[H] by EWS Cell Line TC205 in Presence of SR-B 1 Antibody. FIG. 12C—Uptake of Radioactive Cholesterol Oleate 3[H] by EWS Cell Line CHLA10 in the Presence of SR-B1 Antibody.

FIGS. 13A-13B: FIG. 13A—Comparative IC₅₀ Values for EWS Cell Line (A673) and Cardiomyocytes (H9c2) in 2D Cell Culture Model. FIG. 13B—Comparative IC₅₀ Values for EWS Cell Line (A673) and Cardiomyocytes (H9c2) in 3D Spheroid Cell Culture Model.

### DETAILED DESCRIPTION OF THE PREFERRED EMBODIMENTS

The present disclosure describes nanoparticles that are useful for the treatment of cancers such sarcomas. In some embodiments, the present disclosure provides enhanced drug delivery via HDL-mimetic nanostructures containing disuccinimidyl suberate (DSS) as a crosslinker. The findings reported in this disclosure were obtained using an amphiphilic peptide model (modified via conjugation of a myristic acid residue to the amino terminal aspartic acid) that self-assembles into drug transporting NPs while retaining key features of endogenous HDL (Raut et al., 2020). These studies describe the macromolecular assembly of the MYR-5A lipo-peptide into nano-micellar structures, and the evaluation of their potential as anti-cancer agents, featuring substantially enhanced therapeutic benefits. Thus, the inventors have developed a drug delivery platform with the capability of tumor selective targeting, utilizing a high-density lipoprotein (HDL) mimetic nano-transporter (Raut et al., 2020) cross-linked with DSS, and displaying affinity toward the scavenger receptor class B type I (SR-B1).

Because MYR-5A NPs mimic the structure and function of circulating HDL, upon assembling into spherical micellar structures they interact with SR-B1 receptor to specifically transport payloads (cholesterol or lipophilic small molecules, including drugs) to cells and tumors that express SR-B1. The present disclosure reports the unique features of this enhanced drug delivery strategy for the anthracycline drug, AD 198, via MYR-5A NPs cross-linked with DSS. The data presented include the physical/chemical characterization of the MYR-5A/AD 198 NPs and their interaction with malignant and non-malignant cells. The findings indicate that the DSS cross-linked MYR-5A/AD 198 nanocomplex represents a promising tool for achieving effective cancer chemotherapy in general and EWS therapy, in particular, while also exhibiting significant cardioprotective effects. Specifically, the encapsulated AD 198 payload was found to be delivered to SR-B1 positive cells, presumably via binding to the SR-B1 receptor. Accordingly, the drug payload delivery is achieved without endocytosis of the NP itself, a unique and important feature of this system, compared to most other nano-delivery approaches. The inventors and others have reported selective tumor delivery by rHDL nanoparticles via the SR-B1 receptor successfully. In the present study, MYR-5A-AD 198 nano-complex has shown similar delivery of the cargo indicating that this formulation could have potential benefit for personalized therapy for EWS, as well as minimizing the cardio toxicity issues.

### I. Cancers

Cancer, known medically as a malignant neoplasm, is a broad group of diseases involving unregulated cell growth. In cancer, cells divide and grow uncontrollably, forming malignant tumors, and invading nearby parts of the body. The cancer may also spread to more distant parts of the body through the lymphatic system or bloodstream. Not all tumors are cancerous; benign tumors do not invade neighboring tissues and do not spread throughout the body. There are over 200 different known cancers that affect humans.

The causes of cancer are diverse, complex, and only partially understood. Many things are known to increase the risk of cancer, including tobacco use, dietary factors, certain infections, exposure to radiation, lack of physical activity, obesity, and environmental pollutants. These factors can directly damage genes or combine with existing genetic faults within cells to cause cancerous mutations. Approximately 5-10% of cancers can be traced directly to inherited genetic defects. Many cancers could be prevented by not smoking, eating more vegetables, fruits and whole grains, eating less meat and refined carbohydrates, maintaining a healthy weight, exercising, minimizing sunlight exposure, and being vaccinated against some infectious diseases.

Cancer cells that may be treated with the compositions according to the embodiments include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

In particular, the compositions disclosed herein may find use in treating sarcomas in a subject (e.g., a human subject). Examples of sarcomas that can be treated using the composition include but are not limited to: Osteosarcoma, Chondrosarcoma, Poorly differentiated round/spindle cell tumors (includes Ewing sarcoma), Hemangioendothelioma, Angiosarcoma, Fibrosarcoma/myofibrosarcoma, Chordoma, Adamantinoma, Liposarcoma (includes the following varieties: atypical lipomatous tumor/well-differentiated liposarcoma, dedifferentiated liposarcoma, myxoid sarcoma, pleomorphic liposarcoma, and myxoid pleomorphic liposarcoma), Leiomyosarcoma, Malignant peripheral nerve sheath tumor, Rhabdomyosarcoma, Synovial sarcoma, Malignant solitary fibrous tumor, Atypical lipomatous tumor, Dermatofibrosarcoma protuberans (includes pigmented varieties), Dermatofibrosarcoma protuberans, fibrosarcomatous, Giant cell fibroblastoma, Malignant solitary fibrous tumor, Inflammatory myofibroblastic tumor, Low-grade myofibroblastic sarcoma, Fibrosarcoma (includes adult and sclerosing epithelioid varieties), Myxofibrosarcoma (formerly myxoid malignant fibrous histiocytoma), Low-grade fibromyxoid sarcoma, Giant cell tumor of soft tissues, Leiomyosarcoma, Malignant glomus tumor, Rhabdomyosarcoma (includes the following varieties: embryonal, alveolar, pleomorphic, and spindle cell/sclerosing), Hemangioendothelioma (includes the following varieties: retiform, pseudomyogenic, and epithelioid), Angiosarcoma of soft tissue, Extraskeletal osteosarcoma, Gastrointestinal stromal tumor, malignant (GIST), Malignant peripheral nerve sheath tumor (includes epithelioid variety), Malignant Triton tumor, Malignant granular cell tumor, Malignant ossifying fibromyxoid tumor, Stromal sarcoma not otherwise specified, Myoepithelial carcinoma, Malignant phosphaturic mesenchymal tumor, Synovial sarcoma (includes the following varieties: spindle cell, biphasic, and not otherwise specified), Epithelioid sarcoma, Alveolar soft part sarcoma, Clear cell sarcoma of soft tissue, Extraskeletal myxoid chondrosarcoma, Extraskeletal Ewing sarcoma, Interdigitating dendritic cell sarcoma, Desmoplastic small round cell tumor, Extrarenal rhabdoid tumor, Perivascular epithelioid cell tumor, not otherwise specified, Intimal sarcoma, Undifferentiated spindle cell sarcoma, Undifferentiated pleomorphic sarcoma, Undifferentiated round cell sarcoma, Undifferentiated epithelioid sarcoma, Undifferentiated sarcoma, not otherwise specified.

### II. Targeted Drug Delivery Nano-Complexes and Formulation Thereof

### A. HDL-Mimetic Nano-Complexes

In some aspects, therapies provided herein may comprise an HDL-mimicking drug delivery system. In some embodiments, drug delivery refers to approaches, formulations, manufacturing techniques, storage systems, and technologies involved in transporting a pharmaceutical compound to its target site to achieve a desired therapeutic effect. Principles related to drug preparation, route of administration, site-specific targeting, metabolism, and toxicity are used to optimize efficacy and safety, and to improve patient convenience and compliance. Drug delivery is aimed at altering a drug's pharmacokinetics and specificity by formulating it with different excipients, drug carriers, and medical devices. There is additional emphasis on increasing the bioavailability and duration of action of a drug to improve therapeutic outcomes.

In some aspects of the present disclosure, the nano-complex drug delivery system comprises a lipo-peptide which assembles into nano-micellar structures. In some embodiments, these structures are formulated to mimic the composition of a high-density lipoprotein (HDL). HDL is a lipoprotein, macromolecular structure composed of multiple proteins which transport all lipids around the body within the water outside cells. They are typically composed of 80-100 proteins per particle (organized by one, two or three apolipoproteins; Apolipoprotein A 1(Apo A1)). HDL particles enlarge while circulating in the blood, aggregating more fat molecules) and transporting up to hundreds of fat molecules per particle. With a size ranging from 5 to 17 nm, HDL is the smallest of the lipoprotein particles. It is the densest, as it contains the highest proportion of protein to lipids. Its most abundant apolipoproteins are apo A-I and apo A-II.

The liver synthesizes these lipoproteins as complexes of apolipoproteins and phospholipid, which resemble cholesterol-free flattened spherical lipoprotein particles, whose NMR structure was recently published; the complexes are capable of picking up cholesterol, carried internally, from cells by interaction with the ATP-binding cassette transporter A1 (ABCA1). A plasma enzyme called lecithin-cholesterol acyltransferase (LCAT) converts the free cholesterol into cholesteryl ester (a more hydrophobic form of cholesterol), which is then sequestered into the core of the lipoprotein particle, eventually causing the newly synthesized HDL to assume a spherical shape. HDL particles increase in size as they circulate through the blood and incorporate more cholesterol and phospholipid molecules from cells and other lipoproteins, such as by interaction with the ABCG1 transporter and the phospholipid transport protein (PLTP).

HDL carries many lipid and protein species, several of which have very low concentrations but are biologically very active. For example, HDL and its protein and lipid constituents help to inhibit oxidation, inflammation, activation of the endothelium, coagulation, and platelet aggregation. All these properties may contribute to the ability of HDL to protect from atherosclerosis, and it is not yet known which are the most important. In addition, a small subfraction of HDL lends protection against the protozoan parasite Trypanosoma brucei. This HDL subfraction, termed trypanosome lytic factor (TLF), contains specialized proteins that, while very active, are unique to the TLF molecule.

Most clinical laboratories use automated homogeneous analytical methods to measure HDL in which lipoproteins containing apo B are blocked using antibodies to apo B, then a colorimetric enzyme reaction measures cholesterol in the non-blocked HDL particles. HPLC can also be used. Subfractions (HDL-2C, HDL-3C) can be measured, but clinical significance of these subfractions has not been determined. The measurement of apo-A reactive capacity can be used to measure HDL cholesterol but is thought to be less accurate. Since the HDL particles have a net negative charge and vary by density & size, ultracentrifugation combined with electrophoresis has been utilized to enumerate the concentration of HDL particles and sort them by size with a specific volume of blood plasma. Concentration and sizes of lipoprotein particles can be estimated using NMR fingerprinting.

In some aspects of the present disclosure, myristoylation may occur at the N-terminus of the HDL-mimicking lipoprotein. N-myristoylation is a post-translational modification that attaches a 14-carbon fatty acid to the N-terminal residue of target proteins and peptides. The modification reaction is catalyzed by the enzyme N-myristoyl-transferase which is a ubiquitous and essential enzyme present in eukaryotes. The modification of a protein N-Terminus with a myristoyl group adds an average mass of 206 daltons, and the myristoleylation (=myristoyl with one double bond) adds a mass of 208 daltons to the target protein.

Many target proteins of N-myristoyl-transferase are crucial components of cell signaling pathways and studies on a variety of N-myristoylated proteins suggest that myristic acid may have different roles when attached to different acceptor proteins. Myristoylation typically promotes membrane binding that is essential for proper protein localization or biological function during cell signaling. Since N-myristoyl-transferase adds the myristoyl group as a post translational modification to targeted proteins or peptides it is a validated therapeutic target in opportunistic infections of humans caused by fungi or parasitic protozoa. N-myristoyl-transferase has also been implicated in carcinogenesis, particularly in colon cancer. Apparently, the enzyme is involved in cell signaling and there is evidence for its upregulation in the early stages of tumor formation. Furthermore, an N-myristoylated 5-mer peptide derived from the simian immunodeficiency virus Nef protein specifically recognizes T cells that were isolated from a rhesus macaque cytotoxic T cell line. As mentioned previously, N-myristoylated peptide 5A (MYR-5A) is an HDL-mimetic peptide that has shown potential in treating sarcomas and other forms of cancer.

In some embodiments, the nano-complexes of the present disclosure may be treated with a cross-linker such as Disuccinimidyl suberate (DSS). DSS is a six-carbon lysine-reactive non-cleavable cross-linking agent. It is a homobifunctional N-hydroxysuccinimide ester formed by carbodiimide-activation of carboxylate molecules, with identical reactive groups at either end. The reactive groups are separated by a spacer comprised of a six-carbon alkyl chain. This reagent is mainly used to form intramolecular cros slinks and preparation of polymers from monomers. It is ideal for receptor ligand cross-linking. DSS is reactive towards amine groups (primary amines) at pH 7.0-9.0. It is membrane permeable, therefore permitting intracellular cross-linking, has high purity, is non-cleavable, and is water-insoluble (it must be dissolved in a polar organic solvent such as DMF or DMSO before addition to sample.) Its reaction specificity, reaction product stability, and lack of reaction by-products make it a commonly used cross-linking agent.

As outlined above, HDL-mimicking nanostructures may target scavenger receptor class B, type I (SR-B1). SR-B1 is an integral membrane protein found in numerous cell types/tissues, including enterocytes, the liver and adrenal gland. It is best known for its role in facilitating the uptake of cholesteryl esters from high-density lipoproteins in the liver. This process drives the movement of cholesterol from peripheral tissues towards the liver, where cholesterol can either be secreted via the bile duct or be used to synthesize steroid hormones. This movement of cholesterol is known as reverse cholesterol transport and is a protective mechanism against the development of atherosclerosis, which is the principal cause of heart disease and stroke.

SR-B1 is crucial in carotenoid and vitamin E uptake in the small intestine. SR-B1 is upregulated in times of vitamin A deficiency and downregulated if vitamin A status is in the normal range. In melanocytic cells SCARB1 gene expression may be regulated by the MITF. Although malignant tumors are known to display extreme heterogeneity, overexpression of SR-B1 is a relatively consistent marker in cancerous tissues. While SR-B1 normally mediates the transfer of cholesterol between HDL and healthy cells, it also facilitates the selective uptake of cholesterol by malignant cells. In this way, upregulation of the SR-B1 receptor becomes an enabling factor for self-sufficient proliferation in cancerous tissue. SR-B1 mediated delivery has also been used in the transfection of cancer cells with siRNA, or small interfering RNAs. This therapy causes RNA interference, in which short segments of double stranded RNA acts to silence targeted oncogenes post-transcription. SR-B1 mediation reduces siRNA degradation and off-target accumulation while enhancing delivery to targeted tissues.

In some embodiments, the nanomicelle may be combined with sphingomyelin. Sphingomyelin is a type of sphingolipid found in animal cell membranes, especially in the membranous myelin sheath that surrounds some nerve cell axons. It usually consists of phosphocholine and ceramide, or a phosphoethanolamine head group; therefore, sphingomyelins can also be classified as sphingophospholipids. In humans, sphingomyelins represents —85% of all sphingolipids, and typically make up 10-20 mol % of plasma membrane lipids.

Sphingomyelin consists of a phosphocholine head group, a sphingosine, and a fatty acid. It is one of the few membrane phospholipids not synthesized from glycerol. The sphingosine and fatty acid can collectively be categorized as a ceramide. This composition allows sphingomyelin to play significant roles in signaling pathways: the degradation and synthesis of sphingomyelin produce important second messengers for signal transduction.

Ideally, sphingomyelin molecules are shaped like a cylinder, however many molecules of sphingomyelin have a significant chain mismatch (the lengths of the two hydrophobic chains are significantly different). The hydrophobic chains of sphingomyelin tend to be much more saturated than other phospholipids. The main transition phase temperature of sphingomyelins is also higher compared to the phase transition temperature of similar phospholipids, near 37° C. This can introduce lateral heterogeneity in the membrane, generating domains in the membrane bilayer.

Sphingomyelin undergoes significant interactions with cholesterol. Cholesterol has the ability to eliminate the liquid to solid phase transition in phospholipids. Due to sphingomyelin transition temperature being within physiological temperature ranges, cholesterol can play a significant role in the phase of sphingomyelin. Sphingomyelin are also more prone to intermolecular hydrogen bonding than other phospholipids.

In some embodiments, the nanomicelle may be combined with cholesterol oleate. Cholesterol oleate is a cholesteryl ester, wherein the ester bond is formed between the carboxylate group of a fatty acid and the hydroxyl group of cholesterol. Cholesteryl esters are hydrolyzed by pancreatic enzymes, cholesterol esterase, to produce cholesterol and free fatty acids, and are associated with atherosclerosis.

### B. Anthracyclines

In some aspects, the nano-complexes disclosed herein may transport anthracyclines or anthracycline derivatives. Anthracyclines are a class of drugs used in cancer chemotherapy that are extracted from Streptomyces bacterium. These compounds are used to treat many cancers, including leukemias, lymphomas, breast, stomach, uterine, ovarian, bladder cancer, and lung cancers. The first anthracycline discovered was daunorubicin (Daunomycin), which is produced naturally by Streptomyces peucetius, a species of Actinomycetota. Clinically the most important anthracyclines are doxorubicin, daunorubicin, epirubicin and idarubicin. Doxorubicin in particular, which is sold under the brand name Adriamycin (among others), is an effective chemotherapy treatment used for breast cancer, bladder cancer, Kaposi' s sarcoma, lymphoma, and acute lymphocytic leukemia. Doxorubicin and other anthracyclines are among the most effective anticancer treatments ever developed and are effective against more types of cancer than any other class of chemotherapeutic agents. Their main adverse effect is cardiotoxicity, which considerably limits their usefulness.

N-Benzyladriamycin-14-valerate (AD 198) is an PKC-activating anthracycline which has not shown significant cardiotoxicity, possibly due to the activation of PKC-E, which provides protection against ischemic injury and the development of cardiac hypertrophy (Hofmann et al., 2007). AD 198 is a more hydrophobic doxorubicin derivative that exhibits less topoisomerase II inhibition and DNA binding activity than doxorubicin (He et al., 2005). Administration of AD 198 in rodents caused no significant cardiotoxicity, while administration of doxorubicin resulted in ventricular damage proportionate to dosage (Hofmann et al., 2007).

Anthracyclines act mainly by intercalating with DNA and interfering with DNA metabolism and RNA production. Cytotoxicity is primarily due to inhibition of topoisomerase II after the enzyme induces a break in DNA, preventing relegation of the break and leading to cell death. The basic structure of anthracyclines is that of a tetracyclic molecule with an anthraquinone backbone connected to a sugar moiety by a glycosidic linkage. When taken up by a cell the four-ring structure intercalates between DNA bases pairs while the sugar sits within the minor groove and interacts with adjacent base pairs.

The anthracyclines have been widely studied for their interactions with cellular components and impact on cellular processes. This includes studies in cultured cells and in whole animal systems. A myriad of drug-cellular interactions have been documented in the scientific literature and these vary with respect to the properties of target cells, drug dose and drug intermediates produced. Since artefactual mechanisms of action can be observed, the following mechanisms which occur at clinically relevant drug concentrations are the most important. The chromophore moiety of anthracyclines has intercalating function and inserts in between the adjacent base pair of DNA. The intercalating function inhibits DNA and RNA synthesis in highly replicating cells, subsequently blocking the transcription and replication processes. This is by far the most-accepted mechanism to explain the action of anthracyclines as topoisomerase-II mediated toxicity is evident at clinically relevant drug concentrations. Anthracyclines intercalated into DNA, form a stable anthracycline-DNA-topoisomerase II ternary complex thus “poisoning” the enzyme and impeding the relegation of double-stranded DNA breaks. This topoisomerase-II-mediated DNA damage subsequently promotes growth arrest and recruits DNA repair machinery. When the repair process fails, the lesions initiate programmed cell death.

The quinone moiety of anthracyclines can undergo redox reactions to generate excessive reactive oxygen species (ROS) in the presence of oxidoreductive enzymes such as cytochrome P450 reductase, NADH dehydrogenase and xanthine oxidase. Converting quinone to semiquinone produces free radicals that actively react with oxygen to generate superoxides, hydroxyl radicals and peroxides. In addition, the availability of cellular iron catalyses redox reactions and further generates ROS. The excessive ROS that cannot be detoxified results in oxidative stress, DNA damage, and lipid peroxidation thereby triggering apoptosis. Anthracyclines can also form adducts with DNA by a single covalent bond through an aminal linkage from the 3′-amino of daunosamine to the exocyclic amino of guanine. The supply of extracellular formaldehyde using formaldehyde-releasing prodrugs can promote covalent DNA adduct formation. Such adducts have been shown to block GpC specific transcription factors and induce apoptotic responses.

Results from a recent meta-analysis provide evidence that breast cancer patients with either duplication of centromere 17 or aberrations in TOP2A, the gene coding for topoisomerase-IIα, benefit from adjuvant chemotherapy that incorporates anthracyclines. This does not include subgroups of patients that harbor amplification of HER2. The observations from this study also allow patients to be identified where anthracyclines might be safely omitted from treatment strategies. In the clinic, a maximum recommended cumulative dose is set for anthracyclines to prevent the development of congestive heart failure. As an example, the incidence of congestive heart failure is 4.7%, 26% and 48% respectively when patients received doxorubicin at 400 mg/m², 550 mg/m² and 700 mg/m². Therefore, the lifetime cumulative doxorubicin exposure is limited to 400-450 mg/m² in order to reduce congestive heart failure incidence to less than 5%, although variation in terms of tolerance to doxorubicin exists between individuals. The risk factors that influence the extent of cardiac injury caused by anthracyclines include genetic variability, age (low or high age groups), previous treatments with cardiotoxic drugs and history of cardiac diseases. Children are particularly at risk due to the anthracycline activity that can compromise the development of the immature heart.

Cardiac injury that occurs in response to initial doses of anthracycline can be detected by a rise in troponin level immediately after administration. Biopsy also allows early detection of cardiac injury by evaluating heart ultrastructure changes. Receiving cumulative doses of anthracycline causes left ventricle dysfunction and with continued dosage reaches a certain threshold that can be clinically detected by non-invasive techniques such as 2D echocardiography and strain rate imaging. Advances in developing more sensitive imaging techniques and biomarkers allow early detection of cardiotoxicity and allow cardioprotective intervention to prevent anthracycline-mediated cardiotoxicity.

The predominant susceptibility of the heart to anthracyclines is due in part to a preferential mitochondrial localization of anthracyclines. This is attributed to high affinity interaction between anthracyclines and cardiolipin, a phospholipid present in the heart mitochondrial membrane, as heart tissue contains a relatively high number of mitochondria per cell. Heart tissue also has an impaired defense against oxidative stress, displaying a low level of antioxidant enzymes such as catalase and superoxide dismutase for detoxifying anthracycline-mediated ROS. The mechanisms accounting for anthracycline-induced cardiac damage are complex and interrelated. It was first recognized to be related to the oxidative stress induced by anthracyclines. A more recent explanation has emerged, in which anthracycline-mediated cardiotoxicity is due to anthracycline-topoisomerase IIb poisoning, leading to downstream oxidative stress.

When anthracyclines are given intravenously, it may result in accidental extravasation at injection sites. It is estimated that the extravasation incidence ranges from 0.1% to 6%. Extravasation causes serious complications to surrounding tissues with the symptoms of tissue necrosis and skin ulceration. Dexrazoxane is primarily used to treat anthracyclines post-extravasation by acting as a topoisomerase II inhibitor as well as a chelating agent to reduce oxidative stress caused by anthracyclines. Dexrazoxane has also been used with success as a cardioprotective compound in combination with doxorubicin in metastatic breast cancer patients who have been treated with more than 300 mg/m² doxorubicin, as well as in patients who are anticipated to have a beneficial effect from high cumulative doses of doxorubicin.

Studies of the cardioprotective nature of dexrazoxane provide evidence that it can prevent heart damage without interfering with the anti-tumour effects of anthracycline treatment. Patients given dexrazoxane with their anthracycline treatment had their risk of heart failure reduced compared to those treated with anthracyclines without dexrazoxane. There was no effect on survival though. Radiolabeled doxorubicin has been utilized as a breast cancer lesion imaging agent in a pilot study. This radiochemical, ^{99m}Tc-doxorubicin, localized to mammary tumor lesions in female patients, and is a potential radiopharmaceutical for imaging of breast tumours. In some cases, anthracyclines may be ineffective due to the development of drug resistance. It can either be primary resistance (insensitive response to initial therapy) or acquired resistance (present after demonstrating complete or partial response to treatment). Resistance to anthracyclines involves many factors, but it is often related to overexpression of the transmembrane drug efflux protein P-glycoprotein (P-gp) or multidrug resistance protein 1 (MRP1), which removes anthracyclines from cancer cells. A large research effort has been focused in designing inhibitors against MRP1 to re-sensitize anthracycline resistant cells, but many such drugs have failed during clinical trials.

Liposomal formulations of anthracyclines have been developed to maintain or even enhance the therapeutic efficacy of anthracyclines while reduce its limiting toxicities to healthy tissues, particularly cardiotoxicity. Currently, there are two liposomal formulations of doxorubicin available in the clinics. Doxorubicin is encapsulated in a nano-carrier known as Stealth or sterically stabilized liposomes, consisting of unilamellar liposomes coated with hydrophilic polymer polyethylene glycol (PEG) that is covalently linked to liposome phospholipids. The PEG coating serves as a barrier from opsonization, rapid clearance while the drug is stably retained inside the nano-carriers via an ammonium sulphate chemical gradient. A major advantage of using nano-carriers as a drug delivery system is the ability of the nano-carriers to utilize the leaky vasculature of tumors and their impaired lymphatic drainage via the Enhanced Permeability and Retention (EPR) effect.

The maximum plasma concentration of free doxorubicin after Doxil (a commercial liposomal formulation of doxorubicin) administration is substantially lower compared to conventional doxorubicin, providing an explanation for its low cardiotoxicity profile. However, Doxil can cause Palmar-plantar erythrodysesthesia (PPE, hand and foot syndrome) due to its accumulation in the skin. Doxil has lower maximum tolerable dose (MTD) at 50 mg/m² every 4 weeks compared to free doxorubicin at 60 mg/m² every 3 weeks. Despite this, the maximum cumulative dose for Doxil is still higher compared to doxorubicin due to its cardioprotective characteristics. Myocet is another non-pegylated liposome encapsulated doxorubicin citrate complex approved for use in combination with cyclophosphamide in metastatic breast cancer patients as first line treatment in Europe and Canada. Doxorubicin is loaded into the liposomes just before administration to patients with a maximum single dose of 75 mg/m² every 3 weeks. Myocet has similar efficacy as conventional doxorubicin, while significantly reducing cardiac toxicity.

Drug interactions with anthracyclines can be complex and might be due to the effect, side effects, or metabolism of the anthracycline. Drugs which inhibit Cytochrome P450 or other oxidases may reduce clearance of anthracyclines, prolonging their circulating half-life which can increase cardiotoxicity and other side effects. As they act as antibiotics anthracyclines can reduce the effectiveness of live culture treatments such as Bacillus Calmette-Guerin therapy for bladder cancer. As they act as myelosuppressors anthracyclines can reduce the effectiveness of vaccines by inhibiting the immune system.

Several interactions are of particular clinical importance. Though dexrazoxane can be used to mitigate cardiotoxicity or extravasation damage of anthracyclines it also may reduce their effectiveness and the recommendation is not to start dexrazoxane treatment upon initial anthracycline treatment. Trastuzumab (a HER2 antibody used to treat breast cancer) may enhance the cardiotoxicity of anthracyclines although the interaction can be minimized by implementing a time interval between anthracycline and trastuzumab administration. Taxanes (except docetaxel) may decrease anthracycline metabolism, increasing serum concentrations of anthracyclines. The recommendation is to treat with anthracyclines first if combination treatment with Taxanes is required.

### III. Definitions

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. When used in other contexts, the term “about” is used to indicate a value of ±10% of the reported value, preferably a value of ±5% of the reported value. It is to be understood that, whenever the term “about” is used, a specific reference to the exact numerical value indicated is also included.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### IV. Therapies

### A. Formulations and Routes of Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Aqueous compositions of the present disclosure comprise an effective amount of the composition to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Reference media are, e.g., liquids occurring in “in vivo” methods, such as blood, lymph, cytosolic liquids, or other body liquids, or, e.g., liquids, which may be used as reference media in “in vitro” methods, such as common buffers or liquids. Such common buffers or liquids are known to a skilled person.

In some embodiments, microfluidic devices may be employed to construct the nanomicellar structure. Microfluidics refers to the behavior, precise control, and manipulation of fluids that are geometrically constrained to a small scale (typically sub-millimeter) at which surface forces dominate volumetric forces. Typically, microfluidic systems transport, mix, separate, or otherwise process fluids. Various applications rely on passive fluid control using capillary forces, in the form of capillary flow modifying elements, akin to flow resistors and flow accelerators. In some applications, external actuation means are additionally used for a directed transport of the media. Examples are rotary drives applying centrifugal forces for the fluid transport on the passive chips. Active microfluidics refers to the defined manipulation of the working fluid by active (micro) components such as micropumps or microvalves. Micropumps supply fluids in a continuous manner or are used for dosing. Microvalves determine the flow direction or the mode of movement of pumped liquids. Often, processes normally carried out in a lab are miniaturized on a single chip, which enhances efficiency and mobility, and reduces sample and reagent volumes. In some embodiments, microfluidic processes may be implemented using a Nanoassemblr instrument. A Nanoassemblr can synthesize nanoparticles and formulations thereof for the targeted delivery of therapeutic agents to cells and tissues.

### B. Methods of Treatment

The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the cancer and other drugs being administered concurrently. A composition as described herein is typically administered at a dosage that induces death of cancerous cells (e.g., induces apoptosis of a cancer cell), as assayed by identifying a reduction in hematological parameters (Complete blood count (CBC)), or cancer cell growth or proliferation. In some embodiments, the amount is administered at 200 mg/day, 400 mg/day, 600 mg/day, or 800 mg/day. In some embodiments, those amounts are reduced when the patient is a child. In such embodiments, the dosing is 170 mg/m² or 340 mg/m² per day. In some embodiments, these dosings may be reduced or increased based upon the biological factors of a particular patient such as increased or decreased metabolic breakdown of the drug or decreased uptake by the digestive tract if administered orally. Additionally, new derivatives may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or LSC analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., sarcoma) in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In some embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

Alternatively, but also within the scope of the disclosure, a kit is provided which comprises a first kit component comprising at least one cationic peptide or polymer, at least one lipidoid, and at least one therapeutic agent, formulated, e.g., as a sterile solid or liquid formulation, said first kit component optionally comprising at least one other component as defined herein, such as the pharmaceutical carrier or vehicle; and a second kit component comprising a liquid carrier for dissolving or dispersing the content of the first kit component such as to obtain a composition of the disclosure as described above. Again, the kit components are preferably provided in sterile form, whether solid or liquid, and each of them may comprise one or more additional excipient, or inactive ingredient.

In case the kit or kit of parts comprises a plurality of therapeutic agents, one component of the kit can comprise only one, several or all therapeutic agents comprised in the kit. In an alternative embodiment every/each therapeutic agent may be comprised in a different/separate component of the kit such that each component forms a part of the kit. Also, more than one therapeutic agent may be comprised in a first component as part of the kit, whereas one or more other (second, third etc.) components (providing one or more other parts of the kit) may either contain one or more than one therapeutic agent, which may be identical or partially identical or different from the first component.

Optionally, any of the kit components described above are formulated to represent concentrates, whether in solid or liquid form, and may be designed to be diluted by a biocompatible or physiologically tolerable liquid carrier which may optionally not part of the kit, such as sterile saline solution, sterile buffer, or other solutions that are frequently used as liquid diluents for injectable drugs.

In this context of injectable formulations, the expression “liquid carrier” typically means a well-tolerated aqueous injectable liquid composition having a physiologically acceptable composition, pH and osmolality.

The kit or kit of parts may furthermore contain technical instructions with information on the administration and dosage of the nucleic acid sequence, the inventive pharmaceutical composition or of any of its components or parts, e.g., if the kit is prepared as a kit of parts.

The nanoparticles, the kit and the composition as described above are particularly useful to deliver therapeutic agents to living cells. This may serve a scientific research purpose, a diagnostic application or a therapy. In one of the preferred embodiments, the composition is used as a medicament.

As used herein, a “medicament” means any compound, material, composition or formulation which is useful for the prophylaxis, prevention, treatment, cure, palliative treatment, amelioration, management, improvement, delay, stabilization, or the prevention or delay of reoccurrence or spreading of a disease or condition, including the prevention, treatment or amelioration of any symptom of a disease or condition.

### C. Combination Therapies

It is very common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with at least one therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, anthracyclines and anthracycline derivatives of the present disclosure may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. The following is a general discussion of cancer therapies that may be used in combination with the compositions of the present disclosure.

### 1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids , e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

### 2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects. Moreover, antibodies against any of these compounds can be used to target the anti-cancer agents discussed herein.

Examples of immunotherapies currently under investigation or in use are immune adjuvants, e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, βand γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Hanibuchi et al., 1998; U.S. Pat. No. 5,824,311). It is contemplated that one or more anti-cancer therapies may be employed with the gene silencing therapies described herein.

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancer.

### V. EXAMPLES

All the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1: Preparation and Characterization of Drug Delivery Nano-Complexes (NCs)

Preparation of HDL-Mimetic NCs. AD 198-loaded MYR-5A NPs were prepared as described earlier (Raut et al., 2020), using a microfluidic technology-based benchtop Nanoassemblr instrument, Precision Nanosystem, Vancouver, BC, Canada. MYR-5A and drug/dye stock solutions were dissolved in PBS (pH 7.4) and ethanol respectively. PBS and ethanol solutions were then injected into the separate ports of the microfluidic mixer at a 3:1 water:ethanol ratio, respectively, with a combined flow rate of 7 ml/min. The resultant mixtures were then dialyzed against PBS overnight in 6-8 KD MWCO Spectrum Laboratories dialysis tubing to remove any free drug.

MYR-5A-AD 198 complexes were further processed, to avoid progressive loss of payload, using different cross linkers. Glutaraldehyde, PEG:PE and DSS were added to the formulation of MYR-5A-AD 198 obtained from the Nanoassemblr, at the concentrations of 100 μM, 10 μM and 1.129 mM respectively. The resultant formulations were incubated at room temperature for 30 minutes and the reaction of glutaraldehyde was stopped by addition of 1M lysine (to achieve a final concentration of 100 mM). The reaction with DSS was stopped by Tris buffer, pH 7.4 (via achieving a final concentration of 50 mM). The respective reaction mixtures were dialyzed as mentioned above. The nano-complex with sphingomyelin was formed by evaporating 0.61 mM sphingomyelin from stock in a separate vial. To this, the MYR-5A-AD 198 formulation from Nanoassemblr was added. The mixture was mixed thoroughly and incubated at 50° C. for 30 minutes, sonicated at 30 amp for 3 minutes and then dialyzed as mentioned above. For formulations fabricated with Palmitoyl 5A and Oleic 5A peptide with AD 198, the same Nanoassemblr protocol was followed as used for the MYR-5A formulation.

MYR-5A-[3H]-Cholesterol oleate particles were prepared as follows. In a clean glass vial, 0.1875 mg/mL cholesteryl oleate was removed from a stock of cholesteryl oleate in chloroform. To this, 4 μCi of tritiated cholesteryl oleate ([3H]-Cholesterol oleate) was added. The mixture was mixed by gentle pipetting and incubated for 20 minutes at 4° C. The solvent was evaporated under nitrogen. To this preparation, 5 mg/mL MYR-5A was added. The mixture was mixed by vortexing and then divided into two parts. One part was cross-linked with Disuccinimidyl suberate (DSS) by the method described above while the other was kept without the treating it with DSS. These samples were dialyzed against PBS and named as MYR-5A-Ch-O*-DSS and MYR-5A-Ch-O* respectively.

Characterization of the MYR-5A NCs. The particle diameter and zeta potential of the respective nanoparticles was estimated using Malvern Zetasizer. The formulations were diluted with filtered PBS pH 7.4, for these measurements. An average of 100 runs was captured by the machine and the number distribution of average particle size was reported. The zeta potential measurements were performed by dispersing the particles in aqueous solution at 25° C. with a scattering angle of 90°. The experiment was repeated three times, and the results were averaged. In addition, transmission electron microscopy (TEM) was also used to measure the particle size of the nanoparticles in solution. The drops of the samples were deposited on carbon-coated formvar grids followed by staining with 1% uranyl acetate for 1 min. TEM images were obtained using Tecnai™ Spirit electron microscope (EMCF facility in University of Texas Southwestern Medical Center, Dallas, TX).

The AKTA Fast protein liquid chromatography (FPLC) system, Amersham Biosciences was used to analyze all the nanocomplexes using Superose 6B 10/300 size exclusion column, GE Healthcare. Absorbance at 280 nm was monitored during the runs involving samples and standards. A high molecular weight calibration kit, containing known molecular weight proteins (MW 43,000 to 669,000, including ovalbumin, conalbumin, aldolase, ferritin, thyroglobulin) and blue dextran from GE healthcare were used to calibrate the column using 0.1×PBS as the mobile phase.

All steady state anisotropy measurements were performed at room temperature in a 1 cm×1 cm quartz cuvette. Free drug dissolved in DMSO was used as the control. Free drug and all MYR-5A drug formulations were diluted in PBS to achieve equimolar concentrations in the cuvette. Absorption spectra of the free drug and formulations with nanocomplexes were collected using Cary 50 UV-visible spectrophotometer, Varian Inc., Australia. Cary eclipse spectrofluorometer, Varian Inc., Australia was used to collect steady state measurements of excitation, emission, and anisotropy. The samples were excited at 470 nm and their emission was observed from 500 nm-800 nm, using a 495 nm long pass filter on the observation side. Excitation spectra were collected by scanning the samples from 350-590 nm with the emission observation set at 595 nm. Steady-state anisotropy measurements were conducted using manual polarizers for excitation and emission. The anisotropy was calculated using the following formula:

$$r = \frac{{I}_{VV} - {GI}_{VH}}{{I}_{VV} + 2 {GI}_{VH}}$$

Where, r is the measured anisotropy, G is the instrument correction factor. IVV is the fluorescence intensity measured with vertically oriented polarizers on both the excitation and observation, IVH is the fluorescence intensity measured with vertically oriented polarizer on excitation and horizontal polarizer orientation on the observation.

### Example 2: Drug Release Study

Drug release study was conducted by placing the drug carrying NPs inside the dialysis bag (6-8 KD MWCO) which was placed in a beaker containing 5% BSA to absorb the released free AD 198, while stiffing (400 rpm) at 37° C. Samples were withdrawn from the BSA solution (outside the dialysis bag) to estimated drug release from the NPs at different time points while replacing the BSA solution to maintain the sink conditions.

In vitro distribution of AD 198 in human plasma components. In a glass vial, 0.8 mL of human plasma was mixed with 0.2 mL of MYR-5A-AD 198 NPs to give a final drug concentration of 0.4 mg per mL. The mixture was incubated for 0 to 3 hr. at 37° C. At 0, 1 and 3 hr time points, 0.2 mL were loaded on a Superose 6B Fast Protein Liquid Chromatography (FPLC) column and the absorbance at 280 nm was monitored. Fractions were analyzed by measuring the fluorescence of AD 198 at excitation 490 nm and emission 600 nm. The column was calibrated with HDL, LDL and serum albumin prior to conducting the experiment. In addition, the chromatogram for plasma without drug was recorded as a control.

Determination of critical micellar concentration (CMC) of MYR-5A. The CMC of MYR-5A was determined by the methods of Dominguez et al. (1997). Briefly, 0.5 mg per mL pyrene solution was prepared in methanol and diluted to give a stock concentration of 0.025 mg per mL. In separate tubes, MYR-5A was serially diluted from 2 mg/mL to 0.002 mg/mL by progressively decreasing the MYR-5A concentration by half (tube by tube) To each MYR-5A tube, 50 μL of pyrene stock solution was added. The solution was mixed gently and allowed to stand for 5 minutes at room temperature. Pyrene was excited at 334 nm and its emission was recorded at 373 nm and 384 nm. The fluorescence intensity ratio at 373nm to 384 nm was measured. A graph of the concentration of the MYR-5A vs ratio of fluorescence intensity was plotted, and the CMC was determined.

Cell culture conditions. Ewing Sarcoma cell lines TC 205 and CHLA10, Children's Oncology Group (COG), A673 (Dr Greg Aune, UT Health San Antonio, TX) and Rat Cardiomyocytes H9c2 (ATCC CRL 1446) were cultured according to procedures and culturing conditions provided by COG, Dr Aune and ATCC respectively. Briefly, the cell lines A673 and H9c2 were grown in DMEM, with 10% fetal bovine serum (FBS) and 1% Penicillin streptomycin. TC 205 and CHLA10 cells were grown in IMDM medium with 10% FBS, 1% insulin-transferrin-selenium solution, and 1% Penicillin streptomycin. All the cells were grown by incubating at 37° C. and 5% CO₂. Cells were passaged using 0.25% trypsin to detach the cells from the flasks, once 80%-90% confluency was reached. Spheroid models of the above cell lines was established by using ultralow cluster, ultra-low attachment round bottom 96 well plates, costar, Corning Inc. Initial 5000 cells per well were used for all the cell lines. The cells were grown in respective media for at least 4 days until the spheroids were formed.

Determination of the IC₅₀ values of cell lines using MYR-5A/AD 198 NPs. The respective AD 198 formulations were analyzed using Cell counting kit (CCK-8) kit, Dojindo Molecular Technologies, Tabani, Japan. Briefly, several cell lines were grown according to procedures and culturing conditions provided by the ATCC. Cells density (cell count) was determined using the Cellometer mini, Nexcelom, Lawrence, MA, USA. To initiate cell growth, 5000 cells per well were seeded into 96-well microtiter plates and incubated at 37° C. in 5% CO₂ for 24 hours to allow the cells to attach to the plates. The free drug and the nano-complexes were diluted in DMSO+serum-free medium (SFM) and SFM respectively to yield stock solutions of equivalent molar concentrations. Subsequently, aliquots of the stock solutions were added to microtiter plate wells to achieve the required respective concentration ranges for the cell viability.

Controls included cells without drug, vehicle alone and control without cells with the same formulations for each concentration used. Cells were incubated at 37° C. in 5% CO2 for 48 hours. After incubation, 10 μL of highly water-soluble tetrazolium salt, 2-(2-methoxy-4-nitrophenyl)-3-(4-nitrophenyl)-5-(2,4-disulfophenyl)-2H-tetrazolium, monosodium salt (WST-8) stock solution was added to each well. After 3 hours of incubation at 37° C., the absorbance at 450 nm was measured using a Bio-Tek, Cytation 3 image reader, Agilent, Santa Clara, CA, USA. The concentration required to achieve 50% inhibition of cells growth (IC₅₀), was calculated according to the manufacturer's instructions. Six replicates were used at each concentration. A graph of molar concentration of drug vs Absorbance at 450 nm was plotted. The IC₅₀ value was extrapolated from the graph.

Payload uptake by cells from the MYR-5A NPs. EWS cells A673, TC205 and CHLA9 were plated in 24-well plates (120,000 cells/well) in the respective media with 10% fetal bovine serum (FBS) and incubated at 37° C. with 5% CO₂ for 24 hours. The monolayers were washed with PBS, pH 7.4, and then incubated at 37° C. with serum-free medium for 90 min. Cells were washed with PBS and serum-free medium was again added. The cells were treated with three different dilutions of Anti-Scavenging Receptor SR-B1 antibody (Abcam EPR20190). The plates were incubated for 90 minutes at 37° C. The cells in each well were treated with radiolabeled MYR-5A-Ch-O*-DSS and MYR-5A-Ch-O* formulations. The plates were incubated at 37° C. for 90 minutes and the cells were then washed with 1×PBS, 7.4, followed by 1×PBS pH 3.0 and subsequently again with 1×PBS, pH 7.4, respectively. The washes were carefully collected for radioactive disposal. The cells were then lysed with lysis buffer (50 mM Tris-HCl (pH 8.0), 150 mM sodium chloride, 0.02% sodium azide, 100 mg/ml Phenylmethylsulphonyl fluoride (PMSF), 1 mg/ml aprotinin, and 1% Triton X-100). The lysates were centrifuged at 10,000 rpm for 5 min. The protein content of the lysate was determined by Pierce™ Bicinchoninic acid (BCA) assay, Thermo Scientific, Rockford, IL. The radioactivity ([³H]-Cholesterol oleate) of the samples was measured as counts per minute (CPM) in the Perkin Elmer life Sciences Guardian 1414 liquid scintillation counter. Cells without treatment with SR-B1 antibodies were kept as a control. The uptake of [3H]-Cholesterol oleate was calculated as disintegrations per minute (DPM) per mg protein. Percentage uptake was calculated by assuming that the uptake of [3H]—by the respectively treated cells represented the CE payload uptake from the MYR-5A NPs. The reading obtained with the samples that had been treated with no SR-B1 antibody was taken as 100% uptake.

### Example 3: Characterization of the AD 198 Transporting rHDL Nanoparticles

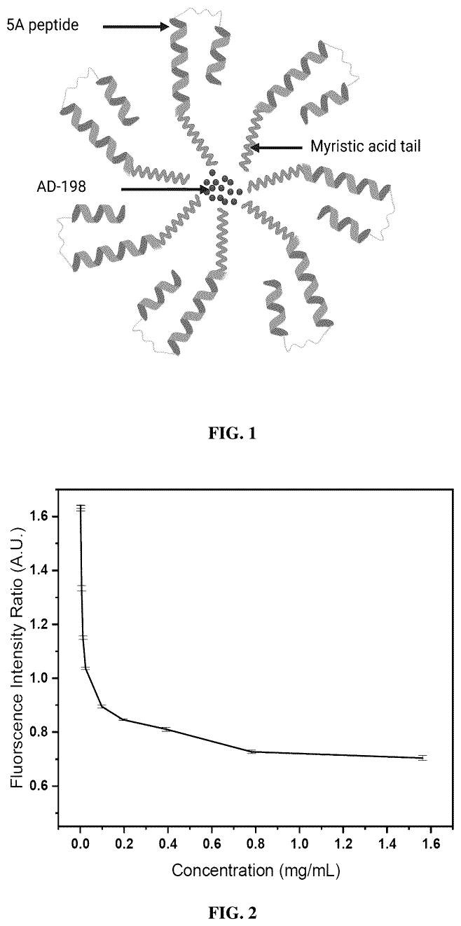

The inventors designed and characterized a number of drug formulations, utilizing basic components of high-density lipoproteins (HDL) to construct the respective rHDL nanocarriers. More recently, they investigated the drug carrying capabilities of a lipo-peptide (MYR-5A) that, upon dissolution in water, has the capability to spontaneously assemble into a drug carrying nanoparticle (FIG. 1).

During the studies reported in this disclosure, the focus of this approach was the description of the mechanism of therapeutic delivery of the drug AD 198 by MYR-5A NPs. The first attempt to characterize the MYR-5A/AD 198 complex was to determine the critical micellar concentration (CMC) of the MYR-5A nanoparticles to indicate the minimal concentration of Myr 5A that can form micelle. The inventors used the method of Dominguez et al. (1997) to determine the CMC as shown in FIG. 2. Based on these studies, they assembled a series of NPs, as previously described, containing Pyrene as a payload with increasing concentration of MYR-5A By plotting a graph of Fluorescence intensity Ratio at 373 to 384 nm vs concentration of Myr 5A

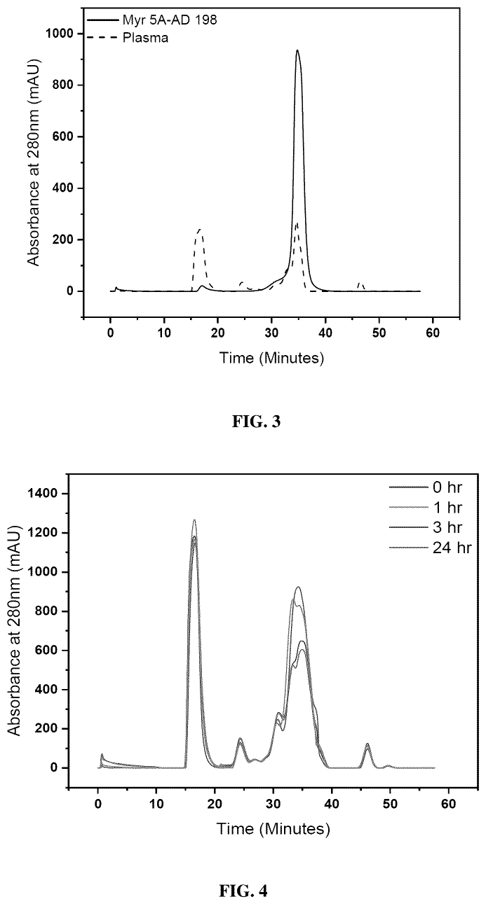

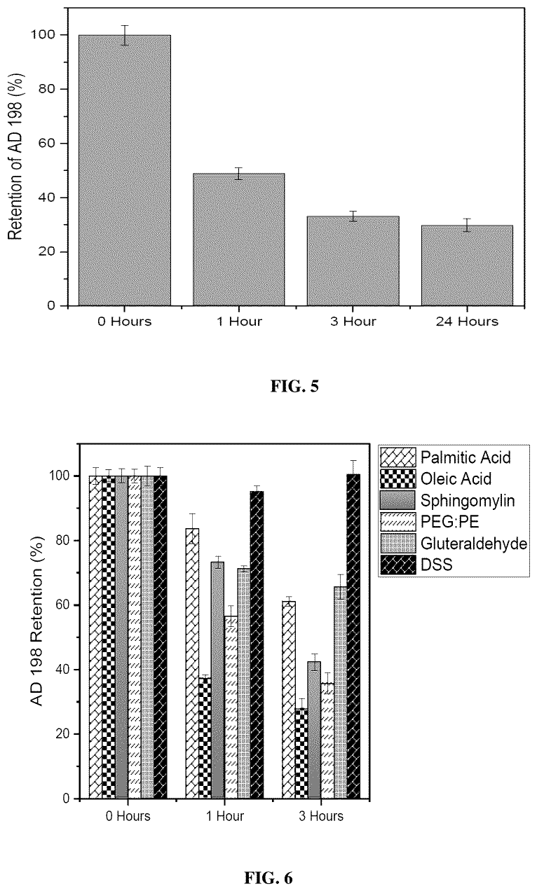

Payload retention of the MYR-5A/AD 198 NPs was assessed by incubating nanoparticles with aliquots of human plasma. First, the data were obtained by recording the Absorbamnce at 280 nm in mAU of the fractions, corresponding to the elution volume upon Fast Protein Liquid Chromatography (FPLC) column for plasma and Myr 5A-AD 198 separately to distinguish different lipoprotein fractions (FIG. 3). Later, Myr 5A-AD 198 NPs were incubated with plasma for 1, 3 and 24 hours (FIG. 4). The data showed the NPs in the HDL fraction of plasma. The data obtained from the plasma incubation studies indicated a progressive, time dependent loss of the AD 198 payload from the MYR-5A NPs as shown by the decrease of the fluorescence from the initial amount (100%) to 30% in 24 hrs (FIG. 5).

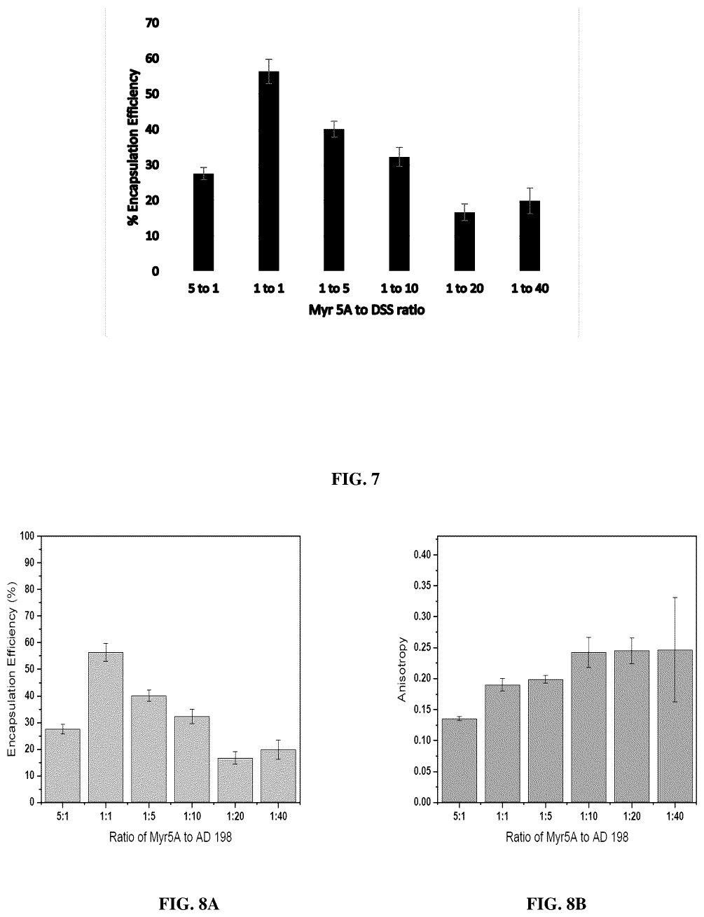

In order to limit or eliminate the payload leakage from the MYR-5A/AD 198 NPs, the inventors explored a number of approaches, including treating the drug loaded NPs with disuccinimidyl-suberate (DSS) to produce crosslinks between the polypeptide chains, via reacting with the epsilon-amino lysine residues. This approach, shown on the extreme right side of the diagram of FIG. 6 turned out to be most successful as it totally prevented the payload leakage, up to 3 hrs, from the NPs as shown below (FIG. 6). Subsequently, the conditions of the crosslinking process were optimized, based on the incorporation efficiency of the AD 198 into the MYR-5A NPs (FIG. 7) by using different ratio of Myr 5A to DSS (a cross Linker). Based on these findings, the formulation with 1:1 ratio of MYR-5A to DSS showed maximum drug encapsulation efficiency and was hence used in all further studies to prepare the stable, cross-linked NPs containing the drug AD 198. Further, the encapsulation efficiency and the fluorescence anisotropy (measured at excitation at 495 nm; emission at 600 nm) (FIGS. 8A-B), indicating the depth of incorporation of the drug molecule within the MYR-5A NPs.

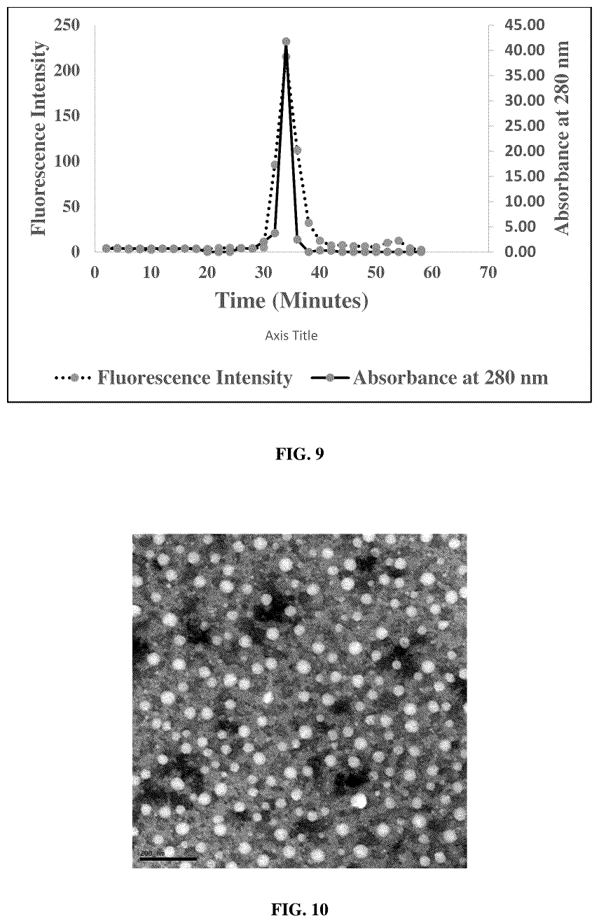

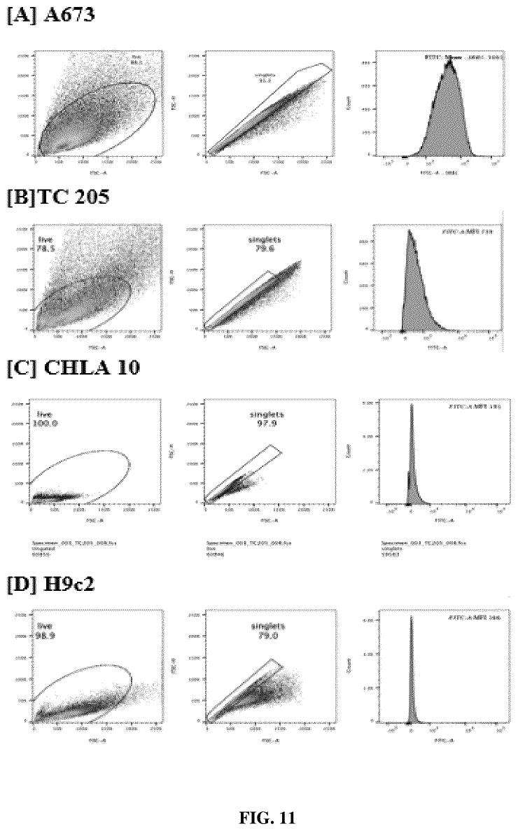

This formulation with cross linker was then loaded on the FPLC column to ensure the position of drug in the NPs and estimate its molecular weight. As shown in FIG. 9, the MYR-5A/AD 198 NPs are represented by very small particles (based on the estimated MW of 66,000), their size corresponding to the pre-beta HDL particles), a feature that could allow their rapid penetration into the tumor microenvironment. The size of the NPs was estimated using TEM and was found to be 7.5±1.1 nm (FIG. 10).

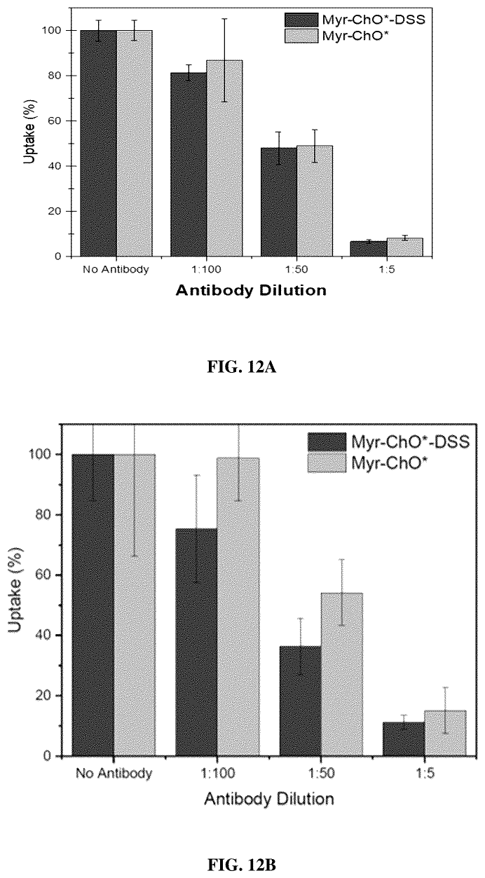

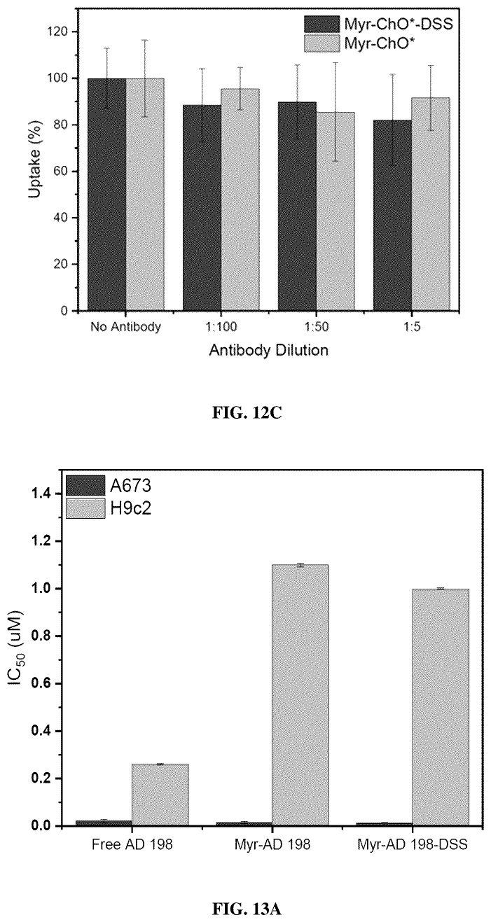

Upon establishing a stable nanocarrier, required for pharmaceutical agents, the inventors focused the studies on the mechanism of the drug payload uptake from the MYR-5A/AD 198 NPs by cancer cells and tumors. The EWS cells exhibited substantial variation with regard to their expression of the SR-B1 receptor. Accordingly, the EWS cell lines A673, TC205 and CHLA 10 were demonstrated to be high, medium and low expressors of SR-B1 receptors (FIG. 11). Next, the inventors addressed the capacity of the biological function of the cross-linked MYR-5A NPs compared to their non-cross-linked counterparts. The data in FIGS. 12A-C. show that the ability of the cross-linked MYR-5A NPs to deliver their lipophilic (AD 198) payload to cancer cells was not impaired or even limited by the cross-linking process. Here the delivery of cholesteryl esters (CE) to different EWS cell lines was shown to be nearly identical between the cross-linked vs the non-cross-linked nanoparticles. However, the drug uptake was directly related to the SR-B1 expression in the cell lines. The data from studies involving CE uptake by these respective cells was consistent with earlier flow cytometry findings (FIG. 11) as revealed by the inhibition of the CE uptake data (presumably via the SR-B1 receptor mechanism) via the SR-B1 antibody (FIGS. 12A-C).

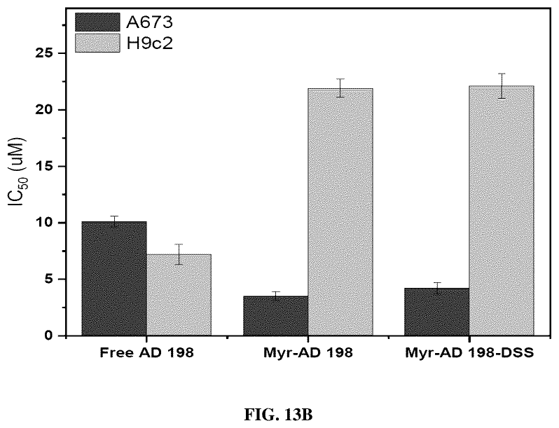

FIGS. 13A-B show the evaluation of the cytotoxicity of the MYR-5A formulation containing AD 198. These studies were carried out with both 2D (cell) and 3D (spheroid) cultures. The data presented earlier shows likewise support the concept that the delivery of anti-cancer agents, to cancer cells and tumors via the MYR-5A NPs, is largely dependent on the SR-B1 receptor. The data shows that in both 2D (FIG. 13A) and 3D (FIG. 13B) models, the sensitivity of malignant (EWS) cells to the MYR-5A/AD 198 formulation was substantially enhanced compared to the normal (cardiac) cells. This occurred, in contrast to the sensitivity that the respective cells exhibited toward the free drug. Once again, cross-linking the AD 198 containing MYR-5A NPs had no effect on the efficiency of the drug delivery to cells. The differential drug delivery based on receptor expression, makes this drug delivery formulation promising for personalized therapy of sarcomas and many other cancers.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims

### VI. REFERENCES

The following references, to the extent that they provide exemplary procedural or other details supplementary to those set forth herein, are specifically incorporated herein by reference.
U.S. Pat. No. 5,801,005
U.S. Pat. No. 5,739,169
U.S. Pat. No. 5,830,880
U.S. Pat. No. 5,846,945
U.S. Pat. No. 5,824,311
Williams et al., Surg. Clin N. Am., 96:1107-1125, 2016.
Anderson et al., Pediatr. Res., 72(2):112-121, 2015.
McGowan et al., Card. Drg. Ther., 31:63-75, 2017.
Edwards et al., BMC Canc., 13:481, 2013.
Solomon et al., Clin. Lymph. Myel., 8(1):21-32, 2008.
Markovic et al., Pharm., 12(11): 1031, 2020.
Yang et al., Jour. Clin. Med., 10(7):1437, 2021.
Bell et al., Sci. Rep., 8:1211, 2018.
Mooberry et al., Front Pharm., 7:466, 2016.
Karimi et al., Ir. Jour. Bas. Med. Sci., 24(8), 2021.
Wilhelm et al., Nat. Rev. Mater., 1:16014, 2016.
Raut et al., Jour. Pharmacol. Exp. Ther,, 373(2):213, 2020.
Lacko et al., Front. Pharmacol., 6:247, 2015.
Meyer et al., Curr. Opin. Lipidol. 24(5):386-392, 2014.
Maugeri et al., Nat. Comm.10:4333, 2019.
Kuai et al., ACS Nano., 10(3):3015-3041, 2016.
McMahon et al., Canc. Treat. Res., 166:129-150, 2015.
Fukuhara et al., PLOS Path., 2014.
Dominguez et al., Jour. Chem. Ed., 74(10):1227, 1997.
Hofmann et al., Jour. Pharmacol. Exp. Ther., 323(2):658-664, 2007.
He et al., Canc. Res., 65(21):10016-10023, 2005.
Hui and Hashimoto, Infec. Immun., 66(11):5329-5336, 1998.
Christodoulides et al., Microbiol., 144(11):3027-3037, 1998.
Bukowski et al., Clin. Canc. Res., 4(10):2337-2347, 1998.
Davidson et al., Jour. Immunother., 21(5):389-398, 1998.
Hellstrand et al., Act. Oncol., 37(4):347-353, 1998.
Qin et al., Proc. Natl. Acad. Sci. USA, 95(24):14411-14416, 1998.
Austin-Ward and Villaseca, Revista Medica de Chile, 126(7):838-845, 1998.
Hanibuchi et al., Int. J. Canc., 78(4):480-485, 1998.

1. A composition comprising a self-assembling nanomicelle comprised of a scavenger receptor class B type 1 (SR-B1) ligand cross-linked with disuccinimidyl suberate (DSS).

2. The composition of claim 1, wherein the ligand is recombinant high-density lipoprotein.

3. The composition of claim 1, wherein in the ligand is myristic acid conjugated-5A peptide.

4. The composition of claim 1, further comprising sphingomyelin and/or cholesterol-oleate.

5. The composition of claim 1, further comprising a therapeutic agent.

6. The composition of claim 5, wherein the therapeutic agent is a small molecule, a peptide or a protein, a nucleic acid, a toxin, or a radioactive compound.

7-10. (canceled)

11. A method of delivering a payload to a cell expressing a scavenger receptor class B type 1 (SR-B1) comprising contacting said cell with a composition according to claim 1.

12. The method of claim 11, wherein the payload is a small molecule such as an anthracycline.

13-15. (canceled)

16. The method of claim 11, wherein the composition is contacted with said cell in vitro.

17. The method of claim 11, wherein the composition is contacted with said cell in vivo.

18. The method of claim 11, wherein, prior to said contacting, said cell exhibits aberrant expression or activity of SR-B1.

19. A pharmaceutical formulation comprising the composition according to claim 1, wherein the formulation further comprises an excipient.

20-24. (canceled)

25. A kit comprising the composition according to claim 1.

26-27. (canceled)

28. The composition according to claim 1, wherein the composition is formulated as a unit dose.

29. The composition according to claim 1, wherein the composition is formulated for systemic administration.

30. The composition according to claim 1, formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

31. A method of treating a disease or disorder in a patient comprising administering to the patient in need thereof a therapeutically effective amount of the composition according to claim 1.

32. The method of claim 31, wherein the disease or disorder is cancer, such as a solid cancer.

33-40. (canceled)

41. The method according to claim 31, wherein the method further comprises a second cancer therapy.

42-47. (canceled)

48. A method of synthesizing the composition of claim 1, the method comprising:
a) contacting the SR-B1 ligand with a therapeutic agent to form a reaction mixture;
b) dialyzing the reaction mixture against a buffer;
c) contacting the SR-B1 ligand with a DSS crosslinker to form a second reaction mixture; and
d) incubating the second reaction mixture for a sufficient time to produce the composition.

49-61. (canceled)


SEQUENCE LISTING (1 sequence)

SEQ ID NO: 1 - protein, 37 aa, synthetic construct
DWLKAFYDKVAEKLKEAFPDWAKAAYDKAAEKAKEAA